(19) United States
(12) Patent Application Publication
(10) Pub. No.: US 2018/0243164 A1
(43) Pub. Date: Aug. 30, 2018

(54) HYDROTHERAPY APPARATUS AND METHOD FOR STERILIZING SAME

(71) Applicant: HIWIN TECHNOLOGIES CORP., TAICHUNG CITY (TW)

(72) Inventors: Hsuan-Tsung CHEN, TAICHUNG CITY (TW); Yen-Nien TIEN, TAICHUNG CITY (TW)

(21) Appl. No.: 15/443,073

(22) Filed: Feb. 27, 2017

(51) Int. Cl. A61H 33/00

(52) U.S. Cl. CPC A61H 33/0087; A61H 2201/105; A61H 2033/0037; A61H 2201/0214

(57) ABSTRACT

A hydrotherapy apparatus, which utilizes the configuration between a sprinkler piping, a reflow piping, a sterilizing piping and a drainage pipe and the operation of a medical motor to control the concentration of a fluid mixture of a medicament and cold water such that each cleaning and sterilizing process does not need to replenish water, saving much time and avoiding waste of water resources. Further, the hydrotherapy apparatus enables the medicament fluid to be maintained at a predetermined concentration to provide a sufficient sterilization effect, and can effectively and completely drain off waste water after the cleaning process, preventing cross-infection between different users.

### BACKGROUND OF THE INVENTION

### 1. Field of the Invention

The present invention relates to hydrotherapy technology and more particularly, to a hydrotherapy apparatus and a method of sterilizing the hydrotherapy apparatus.

### 2. Description of the Related Art

Hydrotherapy mainly involves the utilization of the temperature, buoyancy, pressure and flowing of water for pain relief and treatment, and the application of water jet to increase the speed of water for the purposes of massage and relaxation.

With respect to the related prior art, for example, Japanese Patent No. 4977455 discloses a technique in which bubbles are generated by a bubble generator and mixed with a chemical solution in a bathtub, and the mixed fluid is drawn by a motor back to the pipeline for cleaning. Further, Chinese Patent CN203436239 discloses a technique in which clean water is filled into a bathtub through a water intake valve and a water suction block, and a water pump is operated to pump clean water from the water suction block into the pipeline for cleaning.

In the aforesaid prior art designs, each cleaning process needs to fill clean water into the bathtub to a certain water level, resulting in waste of waiting time and water resources. Further, the waste water cannot be completely drained off after the cleaning process. This is may lead to the risk of cross-infection between different users.

### SUMMARY OF THE INVENTION

The present invention has been accomplished under the circumstances in view. It is the main object of the present invention to provide a hydrotherapy apparatus, which has an automatic cleaning function, and can effectively save waiting time and completely drain off waste water after cleaning.

To achieve this and other objects of the present invention, a hydrotherapy apparatus comprises a water tank, a water spray system and a sterilization system. The water tank comprises a liquid drain. The water spray system is mounted in the water tank, comprising a massage motor, a sprinkler piping, a plurality of spray nozzles, a reflow piping, a drainage pipe and a drainage valve. The sprinkler piping has one end thereof connected to the massage motor. The spray nozzles are mounted in the sprinkler piping. The reflow piping has one end thereof connected to the massage motor, and an opposite end thereof connected to the liquid drain of the water tank. The drainage pipe has one end thereof connected to the reflow piping, and an opposite end thereof connected to the drainage valve. Thus, when the massage motor is turned off, the sprinkler piping allows water to flow therefrom through the massage motor into the reflow piping. When the massage motor is turned on, it pumps water out of the water tank through the reflow piping into the sprinkler piping, enabling water to be sprayed into the water tank by the spray nozzles. This procedure is cycled repeatedly, achieving an effect of reflow.

The sterilization system is disposed adjacent to the water tank, comprising a medicament storage tank holding a medicament, a medical motor, a sterilizing piping, a solenoid valve and a cold water piping. The medical motor is connected to the medicament storage tank. The sterilizing piping has one end thereof connected to the medical motor, and an opposite end thereof connected to the sprinkler piping of the water spray system. The solenoid valve is mounted in the sterilizing piping and adapted for controlling the flow rate of the medicament in the sterilizing piping. The cold water piping has one end thereof connected to the sterilizing piping and an opposite end thereof connected to a water source, and is adapted for filling cold water into the sterilizing piping.

Thus, when the medical motor is turned on, the medical motor pumps the storage medicament from the medicament storage tank to the sterilizing piping for mixing with the cold water being supplied by the cold water piping, enabling the medicament fluid thus obtained to flow from the sterilizing piping to the sprinkler piping of the water spray system to sterilize the sprinkler piping.

Preferably, the reflow piping comprises an oblique pipeline segment and a vertical pipeline segment. The oblique pipeline segment has one end thereof connected to the massage motor, and an opposite end thereof connected to one end of the vertical pipeline segment. The vertical pipeline segment has an opposite end thereof connected to the liquid drain of the water tank. Further, the oblique pipeline segment defines with a bottom wall of the water tank a tilt angle smaller than 10-degrees. The drainage pipe has the one end thereof connected to the junction between the oblique pipeline segment and vertical pipeline segment of the reflow piping. Thus, the tilted design of the oblique pipeline segment of the reflow piping enables water to flow smoothly.

Preferably, when the drainage valve is turned off and the medical motor is turned on, the medicament fluid flows from the sprinkler piping through the massage motor to the oblique pipeline segment of the reflow piping and then flows reversely from the oblique pipeline segment of the reflow piping to the vertical pipeline segment of the reflow piping, achieving an effect of sterilizing the reflow piping.

Preferably, when the drainage valve is turned on and the medical motor is turned off, the water in the water tank is guided in a proper order through the vertical pipeline segment of the reflow piping and the drainage pipe to the outside of the hydrotherapy apparatus, and the cold water in the cold water piping is guided in a proper order through the sterilizing piping, the sprinkler piping, the massage motor, the oblique pipeline segment of the reflow piping and the drainage pipe to the outside of the hydrotherapy apparatus, achieving a cleaning effect after sterilization.

Preferably, the sterilization system further comprises a first check valve mounted in the junction between the medical motor and the sterilizing piping and adapted for preventing the cold water in the cold water piping from flowing into the inside of the medical motor, and a second check valve mounted in the junction between the sterilizing piping and the cold water piping and adapted for preventing the medicament in the sterilizing piping from flowing into the cold water piping.

It is another object of the present invention to provide a method for sterilizing the aforesaid hydrotherapy apparatus. At the first step, turn off the drainage valve and turn on the medical motor, enabling the stored medicament to be delivered from the medicament storage tank to the sterilizing piping to mix with the cold water being supplied by the cold water piping, and also enabling the medicament fluid thus obtained to be delivered from the sterilizing piping to the sprinkler piping where the sprinkler piping is sterilized by the medicament fluid, and then delivered from the sprinkler piping through the massage motor to the reflow piping where the reflow piping is sterilized by the medicament fluid. Thereafter, turn on the drainage valve and turn off the medical motor, enabling the cold water of the cold water piping to flow through the sterilizing piping to the sprinkler piping where the sprinkler piping is cleaned by the cold water, and then to flow from the sprinkler piping through the massage motor to the reflow piping where the reflow piping is cleaned by the cold water, and then to flow from the reflow piping to the outside of the hydrotherapy apparatus via the drainage pipe, achieving an effect of draining off the waste water.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is a schematic system block diagram of a hydrotherapy apparatus in accordance with the present invention.

FIG. 2 corresponds to FIG. 1, illustrating the flow path of the sterilization process.

FIG. 3 corresponds to FIG. 1, illustrating the flow path of the cleaning process.

### DETAILED DESCRIPTION OF THE INVENTION

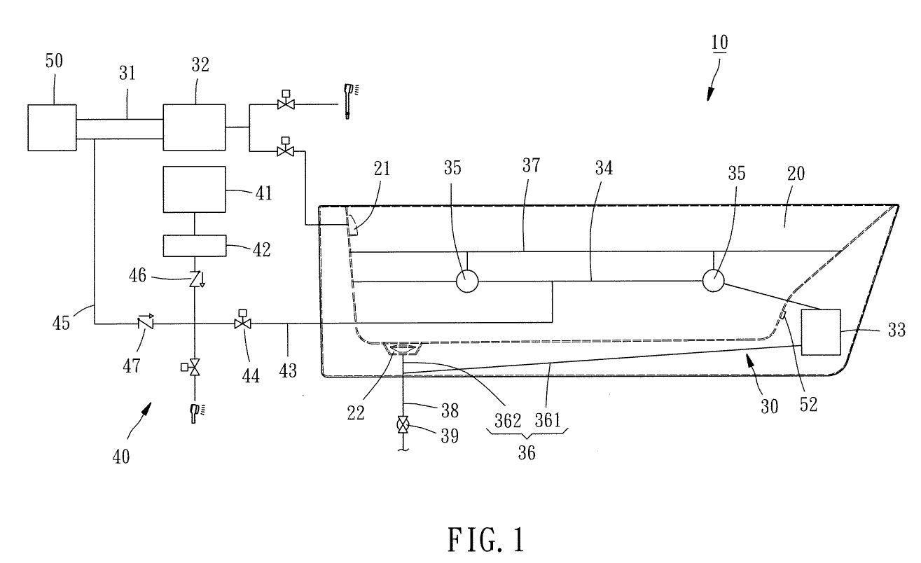

Referring to FIG. 1, a hydrotherapy apparatus 10 in accordance with the present invention is shown. As illustrated, the hydrotherapy apparatus 10 comprises: a water tank 20, a water spray system 30, and a sterilization system 40.

The water tank 20 comprises a water intake port 21 located at a top side thereof, and a liquid drain 22 located at a bottom side thereof.

The water spray system 30 is mounted in the water tank 20, comprising a hot water pipeline 31, and a temperature control unit 32 installed in the hot water pipeline 31. The hot water pipeline 31 has one end thereof connected to a water source 50, and an opposite end thereof connected to the water intake port 21 of the water tank 20. Thus, water provided by the water source can be heated by the temperature control unit 32 and then guided through the hot water pipeline 31 and the water intake port 21 of the water tank 20 into the inside of the water tank 20.

The water spray system 30 further comprises a massage motor 33, a sprinkler piping 34, a plurality of spray nozzles 35, and a reflow piping 36. The sprinkler piping 34 has one end thereof connected to the massage motor 33. The spray nozzles 35 are mounted in the sprinkler piping 34 and spaced from one another along the length of the sprinkler piping 34 for spraying fine water droplets toward the inside of the water tank 20. The reflow piping 36 comprises an oblique pipeline segment 361 and a vertical pipeline segment 362. The oblique pipeline segment 361 defines with the floor a tilt angle α smaller than 10-degrees (preferable 6-degrees), having one end thereof connected to the massage motor 33 and an opposite end thereof connected to a bottom end of the vertical pipeline segment 362. The vertical pipeline segment 362 has an opposite top end thereof connected to the liquid drain 22 of the water tank 20. Thus, when the massage motor 33 is turned on, it pumps water out of the water tank 20 through the reflow piping 36 into the sprinkler piping 35, enabling pumped water to be forced out of the spray nozzles 35 into the water tank 20. At this time, compressed air is delivered through a compressed air piping 37 into water in the water tank 20 to achieve the purpose of massage. When the massage motor 33 is turned off, water in the sprinkler piping 34 flows through the massage motor 33 into the oblique pipeline segment 361 of the reflow piping 36.

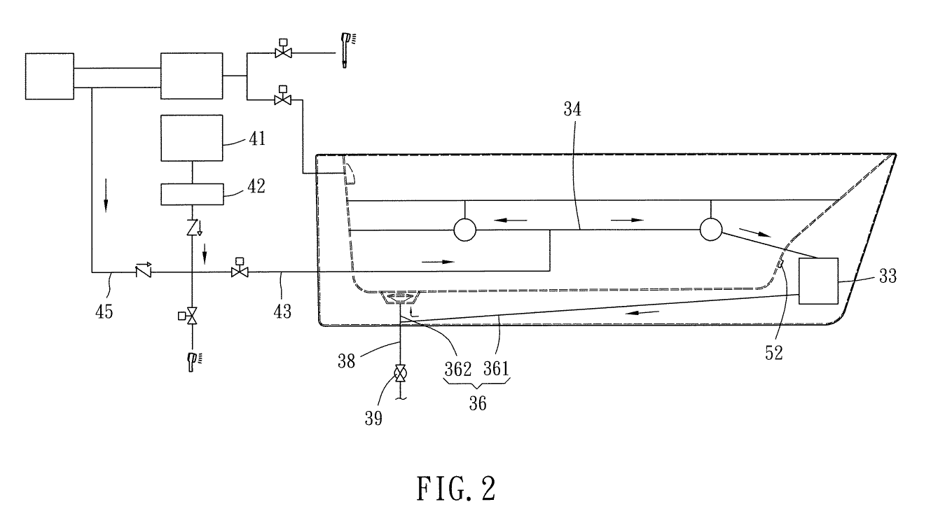

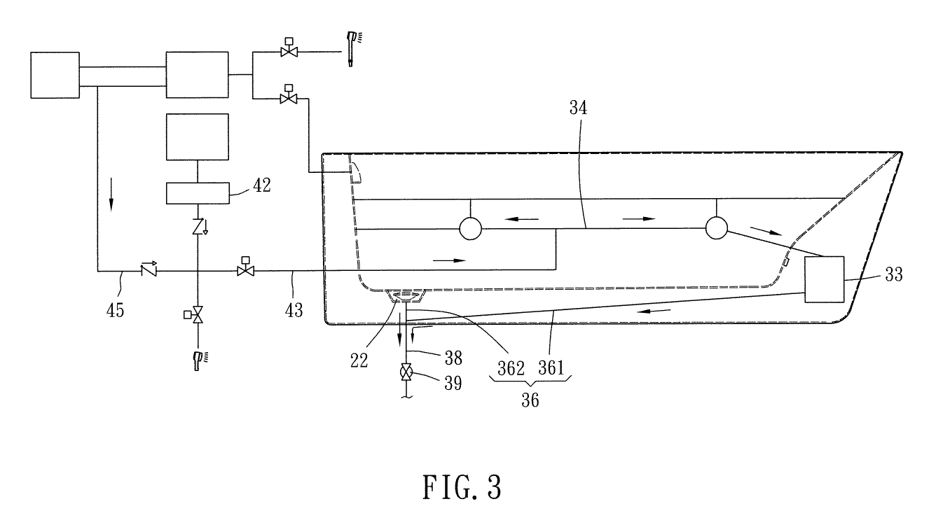

The water spray system 30 further comprises a drainage pipe 38 and a drainage valve 39. The drainage pipe 38 has a top end thereof connected to the junction between the oblique pipeline segment 361 and vertical pipeline segment 362 of the reflow piping 36, and an opposing bottom end thereof connected to the drainage valve 39. Thus, when the drainage valve 39 is turned off, as illustrated in FIG. 2, water in the water tank 20 and the reflow piping 36 is prevented from being discharged through the drainage pipe 38 to the outside of the hydrotherapy apparatus 10, and thus, the water level in the oblique pipeline segment 361 of the reflow piping 36 will rise, causing water to flow reversely into the vertical pipeline segment 362 of the reflow piping 36. When the drainage valve 39 is turned on, as illustrated in FIG. 3, water in the water tank 20 will flow through the liquid drain 22 to the vertical pipeline segment 362 of the reflow piping 36 and then flow from the vertical pipeline segment 362 of the reflow piping 36 to the drainage pipe 38 for discharging to the outside. At this time, water in the oblique pipeline segment 361 of the reflow piping 36 will be discharged through the drainage pipe 38 to the outside.

The sterilization system 40 is disposed adjacent to the water tank 20, comprising a medicament storage tank 41, a medical motor 42, a sterilizing piping 43, a solenoid valve 44 and a cold water piping 45. The medical motor 42 is connected to the medicament storage tank 41, and adapted for pumping the storage medicament out of the medicament storage tank 41. However, in order to accurately control the concentration of the medicament, the medical motor 42 in this embodiment employs a proportional design. The sterilizing piping 43 has one end thereof connected to the medical motor 42, and an opposite end thereof connected to the sprinkler piping 34 of the water spray system 30 for delivering a medicament fluid or cleaning cold water to the sprinkler piping 34. The solenoid valve 44 is installed in the sterilizing piping 43, and adapted for controlling the flow rate of the medicament fluid in the sterilizing piping 43. The cold water piping 45 has one end thereof connected to the sterilizing piping 43 and an opposite end thereof connected to the water source 50, and is adapted for delivering cold water into the sterilizing piping 43 to mix with the medicament into a medicament fluid.

Thus, when the medical motor 42 is turned on, it pumps the storage medicament out of the medicament storage tank 41 into the sterilizing piping 43 for mixing with the cold water of the cold water piping 45 to form a medicament fluid, enabling the medicament fluid thus obtained to be delivered along the sterilizing piping 43 to the sprinkler piping 34 of the water spray system 30 and then the reflow piping 36, thereby sterilizing the sprinkler piping 34 and the reflow piping 36.

The sterilization system 40 further comprises a first check valve 46 and a second check valve 47. The first check valve 46 is mounted in the junction between the medical motor 42 and the sterilizing piping 43, and adapted for preventing the cold water of the cold water piping 45 from flowing to the medical motor 42 to contaminate the medicament. The second check valve 47 is mounted in the junction between the sterilizing piping 43 and the cold water piping 45, and adapted for preventing the medicament fluid of the sterilizing piping 43 from flowing to the cold water piping 45 to contaminate the water source 50.

After understood the structural details of the hydrotherapy apparatus 10, the process of sterilizing the hydrotherapy apparatus 10 is outlined hereinafter. However, it must be noted that the entire cleaning and sterilization process is performed only when the massage motor 33 is turned off.

Step a): Perform sterilization process. As illustrated in FIG. 2, turn off the drainage valve 39 and turn on the medical motor 42, enabling the storage medicament of the medicament storage tank 41 to be delivered by the medical motor 42 to the sterilizing piping 43 where the medicament is mixed with cold water being supplied by the cold water piping 45 at the ratio of 1:200 to form a medicament fluid that immediately flows from the sterilizing piping 43 to the sprinkler piping 34 to sterilize the sprinkler piping 34, and then flows from the sprinkler piping 34 through the massage motor 33 to the reflow piping 36 to sterilize the reflow piping 36. Since the drainage valve 39 has been turned off at this time, the medicament fluid in the oblique pipeline segment 361 of the reflow piping 36 flows to the drainage pipe 38. As soon as the medicament fluid fully fills up the drainage pipe 38, the medicament fluid immediately flows reversely to the vertical pipeline segment 362 of the reflow piping 36 to sterilize the vertical pipeline segment 362.

In the aforesaid step a), if the water level of the water tank 20 is higher than a water level sensor 52 in the water tank 20 before turning on the medical motor 42, turn on the drainage valve 39 at first to discharge water out of the water tank 20 to the extent where the water level in the water tank 20 is below water level sensor 52. If the water level of the water tank 20 is originally below water level sensor 52, the drainage valve 39 will be automatically turned off after turned on the medical motor 42. Further, in order to allow the sprinkler piping 34 to reach the full-pipe sterilizing function without influencing the inflow amount, the sprinkler piping 34 employs ½″ pipes in the present embodiment. Similarly, in order to allow the water flow in the reflow piping 36 smoothly, the reflow piping 36 employs ¾″ pipes in the present embodiment.

Step b): Perform cleaning process after the end of the sterilization process so that the hydrotherapy apparatus 10 can be used by the next user. As illustrated in FIG. 3, turn on the drainage valve 39 and turn off the medical motor 42, enabling the cold water of the cold water piping 45 to flow through the sterilizing piping 43 to the sprinkler piping 34 where the sprinkler piping 34 is cleaned by the cold water, and then to flow out of the sprinkler piping 34 through the massage motor 33 to the oblique pipeline segment 361 of the reflow piping 36 where the oblique pipeline segment 361 is cleaned by the cold water. The waste water after cleaning process is then discharged out of the hydrotherapy apparatus 10 through the drainage pipe 38. At the same time, the water in the water tank 20 flows out of the liquid drain 22 the vertical pipeline segment 362 of the reflow piping 36 to clean the vertical pipeline segment 362, and then flows out of the hydrotherapy apparatus 10 through the drainage pipe 38.

In conclusion, the hydrotherapy apparatus 10 of the present invention utilizes the configuration between the sprinkler piping 34, the reflow piping 36, the sterilizing piping 43 and the drainage pipe 38 to achieve a reflow effect, such that each cleaning and sterilizing process does not need to replenish water in the water tank 20, saving much time and avoiding waste of water resources. Further, using the medical motor 42 to control the concentration of the medicament fluid can provide a sufficient sterilization effect, and the waste water produced after the cleaning process can be effectively and completely discharged off, preventing cross-infection between different users.

What is claimed is:

1. A hydrotherapy apparatus, comprising:
a water tank comprising a liquid drain;
a water spray system mounted in said water tank, said water spray system comprising a massage motor, a sprinkler piping, a plurality of spray nozzles, a reflow piping, a drainage pipe and a drainage valve, said sprinkler piping having one end thereof connected to said massage motor, said spray nozzles being mounted in said sprinkler piping, said reflow piping having one end thereof connected to said massage motor and an opposite end thereof connected to said liquid drain of said water tank, said drainage pipe having one end thereof connected to said reflow piping and an opposite end thereof connected to said drainage valve, said sprinkler piping allowing water to flow therefrom through said massage motor into said reflow piping when said massage motor is turned off, said massage motor being adapted for pumping water out of said water tank through said reflow piping into said sprinkler piping for spraying into said water tank by said spray nozzles when said massage motor is turned on; and
a sterilization system disposed adjacent to said water tank, said sterilization system comprising a medicament storage tank holding a medicament, a medical motor, a sterilizing piping, a solenoid valve and a cold water piping, said medical motor being connected to said medicament storage tank, said sterilizing piping having one end thereof connected to said medical motor and an opposite end thereof connected to said sprinkler piping of said water spray system, said solenoid valve being mounted in said sterilizing piping, said cold water piping having one end thereof connected to said sterilizing piping, said medical motor being adapted for pumping the said medicament out of said medicament storage tank into said sterilizing piping to mix with cold water being provided by said cold water piping, enabling the mixed fluid thus obtained to be delivered from said sterilizing piping to said sprinkler piping of said water spray system.

2. The hydrotherapy apparatus as claimed in claim 1, wherein said reflow piping comprises an oblique pipeline segment and a vertical pipeline segment, said oblique pipeline segment having one end thereof connected to said massage motor and an opposite end thereof connected to one end of said vertical pipeline segment, said vertical pipeline segment having an opposite end thereof connected to said liquid drain of said water tank, said oblique pipeline segment defining with a bottom wall of said water tank a tilt angle smaller than 10-degrees, said drainage pipe has the said one end thereof connected to the junction between said oblique pipeline segment and said vertical pipeline segment of said reflow piping.

3. The hydrotherapy apparatus as claimed in claim 2, wherein when said drainage valve is turned on and said medical motor is turned off, the water in said water tank is guided in a proper order through said vertical pipeline segment of said reflow piping and said drainage pipe to the outside of the hydrotherapy apparatus, and the cold water in said cold water piping is guided in a proper order through said sterilizing piping, said sprinkler piping, said massage motor, said oblique pipeline segment of said reflow piping and said drainage pipe to the outside of the hydrotherapy apparatus.

4. The hydrotherapy apparatus as claimed in claim 3, wherein when said drainage valve is turned off and said medical motor is turned on, the said medicament fluid flows from said sprinkler piping through said massage motor to said oblique pipeline segment of said reflow piping and then flows from said oblique pipeline segment of said reflow piping to said vertical pipeline segment of said reflow piping.

5. The hydrotherapy apparatus as claimed in claim 1, wherein said sterilization system further comprises a first check valve mounted in the junction between said medical motor and said sterilizing piping and adapted for preventing the said cold water in said cold water piping from flowing into the inside of said medical motor, and a second check valve mounted in the junction between said sterilizing piping and said cold water piping and adapted for preventing the said medicament in said sterilizing piping from flowing into said cold water piping.

6. A method for sterilizing the hydrotherapy apparatus as claimed in claim 1, comprising the steps of:
a) turning off said drainage valve and turning on said medical motor for enabling the stored medicament to be delivered from said medicament storage tank to said sterilizing piping to mix with the cold water being supplied by said cold water piping and also enabling the medicament fluid thus obtained to be delivered from said sterilizing piping to said sprinkler piping where said sprinkler piping is sterilized by the said medicament fluid and then delivered from said sprinkler piping through said massage motor to said reflow piping where said reflow piping is sterilized by the said medicament fluid; and
b) turning on said drainage valve and turning off said medical motor for enabling the cold water of said cold water piping to flow through said sterilizing piping to said sprinkler piping where said sprinkler piping is cleaned by the cold water, and then to flow from said sprinkler piping through said massage motor to said reflow piping where said reflow piping is cleaned by the cold water, and then to flow from said reflow piping to the outside of the hydrotherapy apparatus via said drainage pipe.

7. The method for sterilizing the hydrotherapy apparatus as claimed in claim 6, wherein in step a), if the water level of said water tank is higher than a water level sensor in said water tank before turning on said medical motor, turn on said drainage valve at first to discharge water out of said water tank to the extent where the water level in said water tank is below said water level sensor.

8. The method for sterilizing the hydrotherapy apparatus as claimed in claim 6, wherein in step b), the water in said water tank is discharged out of the hydrotherapy apparatus through said liquid drain, said reflow piping and said drainage pipe in a proper order.

9. The method for sterilizing the hydrotherapy apparatus as claimed in claim 6, wherein in step a) the stored medicament of said medicament storage tank is mixed with the cold water being supplied by said cold water piping at a ratio of 1:200.
